(19) United States
(12) Patent Application Publication
(10) Pub. No.: US 2023/0329775 A1
(43) Pub. Date: Oct. 19, 2023

(54) ARTICULATING KNIFE ACTUATOR FOR ELECTROSURGICAL INSTRUMENT

(71) Applicant: Conmed Corporation, Largo, FL (US)

(72) Inventors: Aaron Szabo, Swanton, OH (US); Mason Williams, Centennial, CO (US)

(73) Assignee: Conmed Corporation, Largo, FL (US)

(21) Appl. No.: 18/026,956

(22) PCT Filed: Oct. 5, 2021
(86) PCT No.: PCT/US2021/053546

§ 371 Date: Mar. 17, 2023

Related U.S. Application Data
(60) Provisional application No. 63/087,530, filed on Oct. 5, 2020.

(51) Int. Cl. A61B 18/14

(52) U.S. Cl. CPC A61B 18/1445; A61B 2018/1455; A61B 2018/0063

(57) ABSTRACT

A knife driving assembly that can advance and retract a knife along a knife blade pathway defined by the jaws of the surgical instrument regardless of continuous rotation of the shaft and jaws. The assembly includes a housing having a shaft extending along a longitudinal axis and supporting to a pair of jaws that define a knife pathway and a lever pivotally mounted to housing and extending into the housing. A knife actuator is coupled to the lever at a first end and to a bearing sleeve at a second end. A drive shaft is secured to the bearing sleeve and extends within the shaft to move a knife along the knife pathway in response to movement of the bearing sleeve. As a result, the main shaft, and the drive shaft, may be rotated through 360 degrees with movement of lever capable of moving the knife.

### CROSS-REFERENCE TO RELATED APPLICATIONS

The present application claims priority to U.S. Provisional Application No. 63/087,530, filed on Oct. 5, 2020, hereby incorporated by reference in its entirety.

### BACKGROUND OF THE INVENTION

### 1. Field of the Invention

The present invention relates surgical instruments and, more specifically, to a vessel sealer having an articulated knife actuator for advancing a knife into the jaws of the vessel sealer.

### 2. Description of the Related Art

Electrosurgical vessel sealers are surgical instruments that are used for the occlusion of blood vessels and halting of bleeding during surgical procedures. The electrodes of the vessel sealer are carried by a pair of opposing jaws mounted to the end of an elongated shaft and interconnected to an electrosurgical generator that can selectively supply radiofrequency (RF) energy to the electrodes. A user may close the jaws around a vessel to be sealed by squeezing a lever associated with a handle assembly. The vessel may then be sealed by supplying the RF energy to the clamped vessel and the vessel can be severed by extending a knife blade along tracks formed in the jaws. Conventional approaches to driving the knife blade do not allow for continuous rotation of the shaft. Accordingly, there is a need for an approach that can allow the shaft of the instrument to rotation through 360 degrees while still allowing a user to operate the knife blade once the shaft is positioned as desired.

### BRIEF SUMMARY OF THE INVENTION

The present invention is a knife driving assembly that can advance and retract a knife along a knife blade pathway defined by the jaws of the surgical instrument regardless of continuous rotation of the shaft and jaws. In a first embodiment, a surgical instrument according to the present invention comprises a housing having a shaft extending along a longitudinal axis and supporting to a pair of jaws that define a knife pathway and a lever pivotally mounted to housing and extending into the housing. A knife actuator is coupled to the lever at a first end and extending longitudinally within housing to a second end. A bearing sleeve is coupled to the second end of the knife actuator. A drive shaft is secured to the bearing sleeve for moving a knife along the knife pathway in response to movement of the bearing sleeve. The lever may include a bearing cup mechanically coupling the lever to the knife actuator. The second end of the knife actuator may comprise a fork extending about the bearing sleeve. The bearing sleeve may include a pair of bearing members positioned on either side of the fork of the knife actuator. The shaft and the drive shaft may be rotatable through 360 degrees. Pivotal movement of the level between a first position and a second position causes the knife actuator to drive the bearing sleeve from a retracted position to an extended position. The bearing sleeve may biased toward the first position to retract the knife blade after use.

In another embodiment, the present invention is a method of making a surgical device having a knife blade that can retract and extend within jaws that are free to continuously rotate. The method includes providing a lever pivotally mounted to a housing and extending partially therein. The lever is coupled to a first end of a knife actuator that extends longitudinally within the housing. A second end of the knife actuator is coupled to a bearing sleeve that extends around a rotatable shaft of the surgical device. The knife blade is fixed to the bearing sleeve so that movement of the lever from a first position to a second position advances the knife actuator distally within the housing and drives the bearing sleeve along the shaft to move the knife blade from a retracted position to an extended position within the jaws.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a perspective view of an electrosurgical system having an electrosurgical instrument with a knife actuating assembly according to the present invention;

FIG. 2 is a perspective view of the jaws of electrosurgical instrument that can support a knife driven by a knife actuating assembly according to the present invention;

FIG. 3 is schematic of the lower jaw showing movement of the knife blade from the retracted position to the extending position in response to movement of the knife actuating assembly according to the present invention;

FIG. 4 is a perspective view of a knife actuating assembly according to the present invention;

FIG. 5 is a schematic showing the movement of the knife actuating assembly according to the present invention from a first position to a second position in response to manual movement of the lever;

FIG. 6 is a perspective view of the lever and knife actuator according to the present invention; and

FIG. 7 is a schematic of the coupling of the lever and the knife actuator according to the present invention.

### DETAILED DESCRIPTION OF THE INVENTION

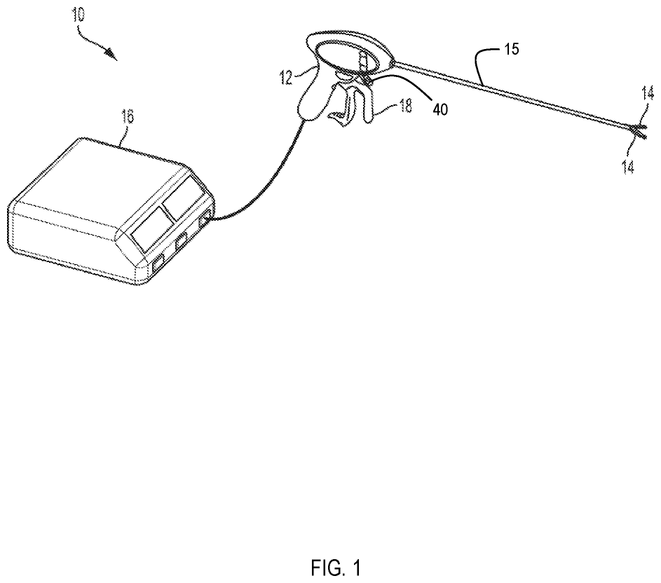

Referring to the figures, wherein like numeral refer to like parts throughout, there is seen in FIG. 1 an electrosurgical system 10 comprising a vessel sealer 12 having a pair of conductive opposing jaws 14 that are interconnected to an electrosurgical generator 16 that can supply RF energy to electrodes of jaws 14 for the desiccation of a blood vessel trapped between jaws 14. As is known in the art, jaws 14 are pivotally mounted to a longitudinally extending shaft 15 of vessel sealer 12 for movement between an open position and a closed position in response to a user operating a lever 18 extending from vessel sealer 12.

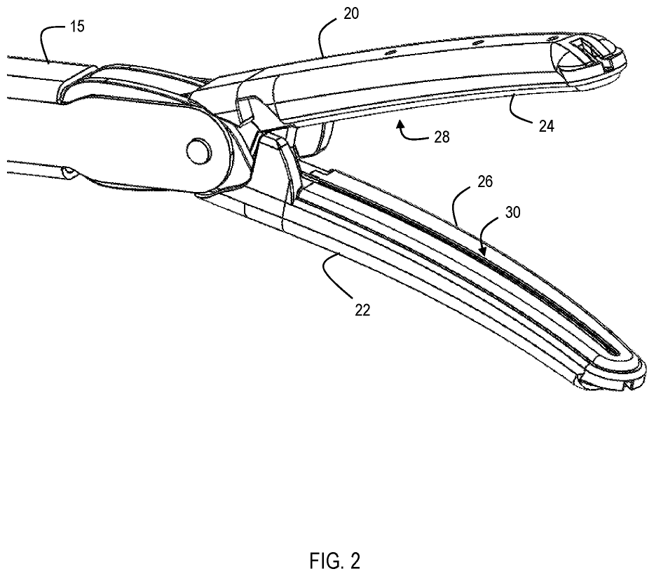

Referring to FIG. 2, jaws 14 are comprised of two opposing jaw members 20 and 22. Jaw members 20 and 22 support inner conductive faces 24 and 26 that are electrically isolated from the rest of jaws 14 and sealer 12 and interconnected to electrosurgical generator 16. Inner conductive faces 24 and 26 have tracks 28 and 30 formed therethrough, respectively, so that when jaw members 20 and 22 are closed, tracks 28 and 30 align to define a knife pathway that intersects the region between jaw members 20 and 22 where tissue is desiccated between jaws 14 when they are energized.

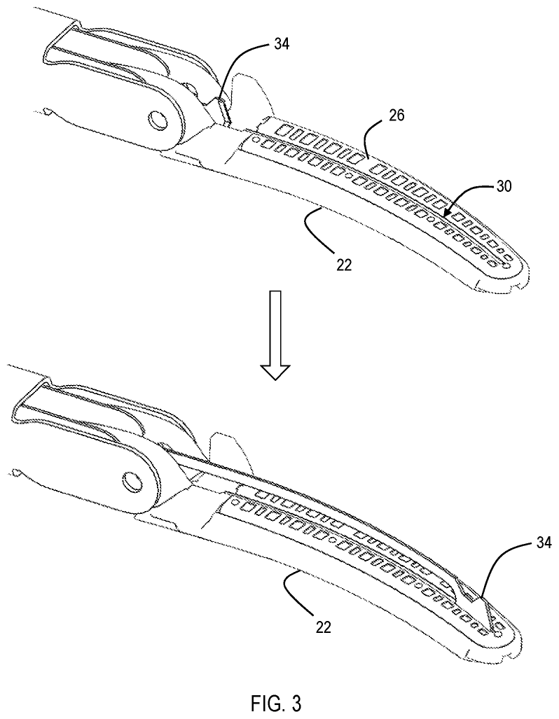

Referring to FIG. 3, a knife blade 34 is aligned with and positioned in the knife pathway defined by tracks 28 and 30 and may be selectively extended through and withdrawn from the knife pathway, such as by a user operating a lever 40 pivotally mounted to the housing 42 of sealer 12, to sever any tissue trapped in jaws 14. As described herein, knife blade 34 may be moved between the retracted position and the extended position regardless of the orientation of the jaws. For example, a user of vessel sealer 12 may rotate shaft 15 through 360 degrees to position jaws 14 is a particular orientation for a procedure without compromising the ability of knife blade 34 to be driven into the extended position to sever any tissue captured in jaws 14.

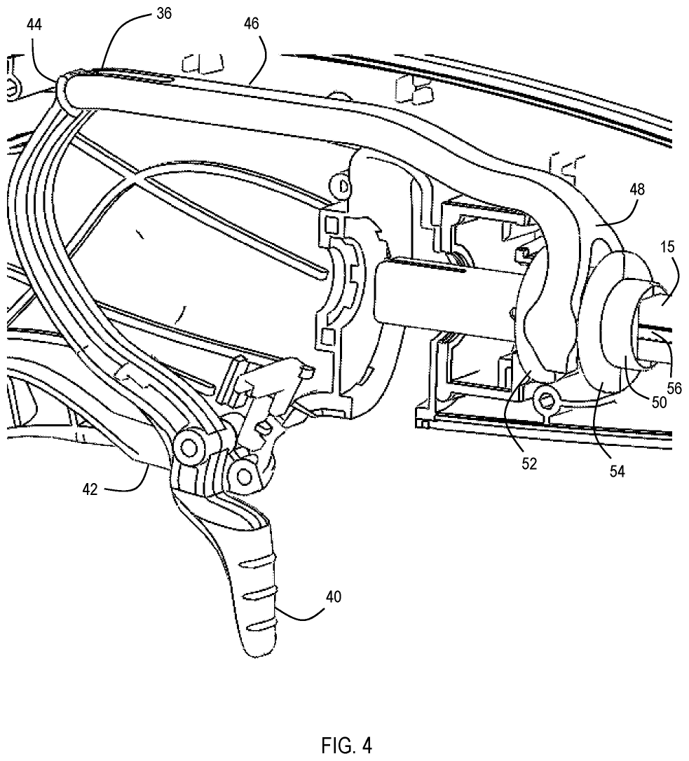

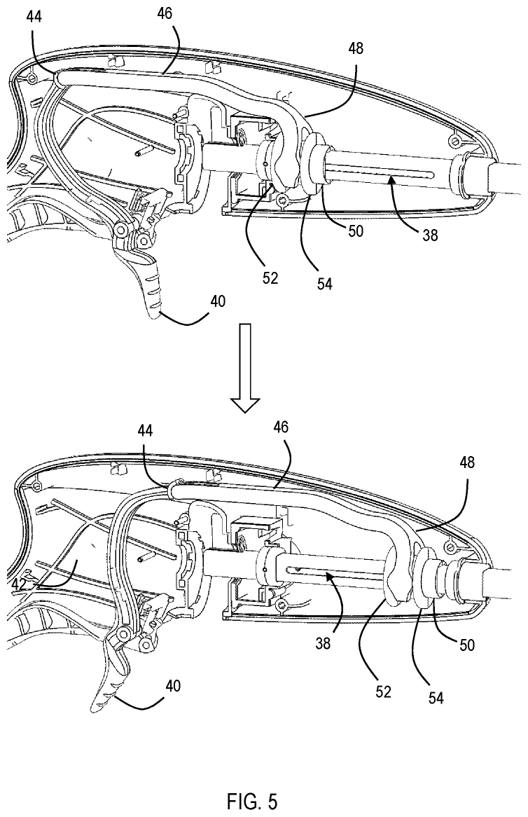

Referring to FIG. 4, a lever 40 is pivotally mounted to the housing 42 and extends into housing 42 to terminate in a bearing cup 44. Bearing cup 44 providing a mechanical connection to a first end 36 of a knife actuator 46 that engages cup 44 and extends longitudinally along the inside of housing 42 to a second end forming a two-armed fork 48 that extends around and is mechanically coupled to a bearing sleeve 50. Bearing sleeve 50 includes opposing bearing members 52 and 54 that are positioned longitudinally about fork 48 so that movement of knife actuator 46 can drive bearing sleeve 50 longitudinally. Bearing sleeve 50 is secured to shaft 15 for rotation therewith. For example, bearing sleeve 50 may have an internal geometry keyed to the outer geometry of shaft 15. Bearing sleeve 50 is secured to a drive shaft 56 that is positioned within and extends inside shaft 15 of vessel sealer 12 and is longitudinally moveable relative thereto. For example, as seen in FIG. 5, shaft 15 includes longitudinal slots 38 permitting bearing sleeve 50 to be secured to drive shaft 56, while allowing bearing sleeve 50 to translate longitudinally along shaft 15. Drive shaft 56 is fixed to knife blade 34 so that longitudinal movement of bearing sleeve 50 along shaft 15 will push drive shaft 56 longitudinally and thus move knife 34 along the knife pathway of jaws 14. Shaft 15 is mounted to housing 42 for continuous rotation about its longitudinally and jaws 14 are fixed relative to shaft, thereby allowing a user to position jaws 14 as desired by rotating shaft 15. Bearing sleeve 50 is capable of moving drive shaft 56 longitudinally regardless of the rotational positioning of bearing sleeve 50 along with shaft 15.

Referring to FIG. 5, pivotal movement of the exposed end of lever 40 proximally toward housing 42 will drive knife actuator 46 so that knife actuator 46 pushes bearing sleeve 50 distally, thereby causing shaft 56 to drive a knife blade coupled to shaft longitudinally along the knife pathway of jaws 14 to sever any tissue trapped therein. Lever 40 is thus moveable from a first position to a second position to cause knife actuator 46 to drive bearing sleeve 50 distally along shaft 15, which will move the knife blade from a retracted position to an extended position to sever tissue trapped in jaws 14. As is known in the art, bearing sleeve 50 may be biased to return to the retracted position in the absence of a user force being applied to lever 40, thereby withdrawing knife from the extended position in the knife pathway to the retracted position. Alternatively, a user could manually move lever 40 back to the first position to retract knife 34 from knife pathway.

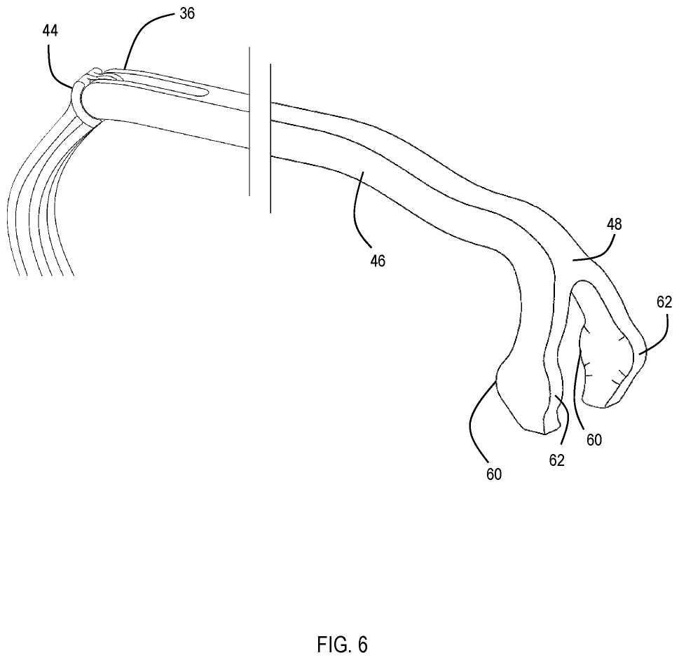

Referring to FIG. 6, each arm of fork 48 has bearing surfaces 60 and 62 that extend proximately and distally to engage bearing members 52 and 54, respectively. Bearing surfaces 60 and 62 allow for the transmission of forces to and from bearing sleeve 50 regardless of rotational positioning of sleeve 50. Bearing surfaces 60 and 62 also for the transmission of forces to and from bearing sleeve 50 when knife actuator 46 is driven by lever 40 as the coupling between lever 40 and knife actuator 46 may produce some pivoting of knife actuator 46 due to the mechanical coupling to bearing cup 44. The combination of lever 40, knife actuator 46, and bearing sleeve 50 allows pivoting of lever 40 translate into longitudinal motion of knife actuator 46 and thus bearing sleeve. In addition, the coupling between fork 48 and bearing sleeve 50 allows the longitudinal movement of knife actuator 46 to drive bearing sleeve 50 to actuate the knife while simultaneously allowing shaft 15 to be rotated through 360 degrees without any loss in the ability to operate the knife.

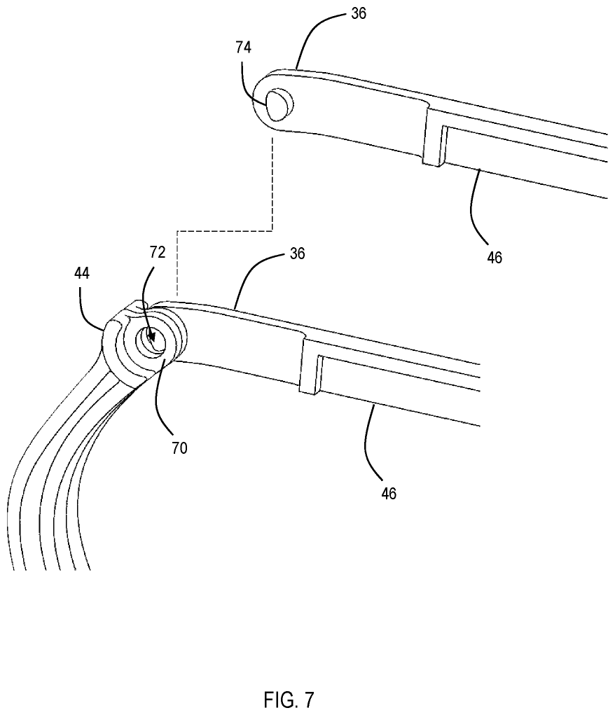

Referring to FIG. 7, bearing cup 44 supports a tab 70 having an aperture 72 formed therethrough. First end 36 of knife actuator 46 is split and includes a post 74 extending transversely to the longitudinal axis of knife actuator 46 to engage aperture 72. Tab 70 of cup 44 and post 74 of knife actuator 46 form a rotational joint that allows pivoting of lever 40 to drive knife actuator 46 longitudinally within housing 42 of vessel sealer 12 and thus operate knife 34.

What is claimed is:

1. A surgical instrument, comprising:
a housing having a shaft extending along a longitudinal axis and supporting to a pair of jaws that define a knife pathway;
a lever pivotally mounted to and extending into the housing;
a knife actuator coupled to the lever at a first end and extending longitudinally within housing to a second end;
a bearing sleeve coupled to the second end of the knife actuator; and
a knife positioned in the knife pathway and coupled to the bearing sleeve for movement therewith in response to movement of the lever and knife actuator.

2. The surgical instrument of claim 1, wherein the lever includes a bearing cup mechanically coupling the lever to the knife actuator.

3. The surgical instrument of claim 2, wherein the second end of the knife actuator comprises a fork extending about the bearing sleeve.

4. The surgical instrument of claim 3, wherein the bearing sleeve includes a pair of bearing members positioned on either side of the fork of the knife actuator.

5. The surgical instrument of claim 4, wherein the shaft is rotatable through 360 degrees.

6. The surgical instrument of claim 5, wherein pivotal movement of the lever between a first position and a second position will cause the knife actuator to drive the bearing sleeve from a retracted position to an extended position.

7. The surgical instrument of claim 6, wherein the bearing sleeve is biased toward the first position.

8. The surgical instrument of claim 7, wherein the bearing cup includes a tab and an aperture formed through the tab.

9. The surgical instrument of claim 8, wherein the first end of the knife actuator includes a post engaged with the aperture of the tab of the bearing cup.

10. A method of making a surgical device having a knife blade that can retract and extend within jaws that are free to continuously rotate, comprising:
providing a lever pivotally mounted to a housing and extending partially therein;
coupling the lever to a first end of a knife actuator that extends longitudinally within the housing;
coupling a second end of the knife actuator to a bearing sleeve that extends around a rotatable shaft of the surgical device; and
fixing the knife blade to the bearing sleeve so that movement of the lever from a first position to a second position advances the knife actuator distally within the housing and drives the bearing sleeve along the shaft to move the knife blade from a retracted position to an extended position within the jaws.

11. The method of claim 10, wherein the lever includes a bearing cup that is used to mechanically couple the lever to the knife actuator.

12. The method of claim 11, wherein the second end of the knife actuator is coupled to the bearing sleeve by a fork that extends about the bearing sleeve.

13. The method of claim 12, wherein the bearing sleeve includes a pair of bearing members positioned on either side of the fork of the knife actuator.
